(19) United States
(12) Patent Application Publication
(10) Pub. No.: US 2021/0022967 A1
(43) Pub. Date: Jan. 28, 2021

(54) Multi-component oral care formulation and multi-compartment delivery systems thereof

(71) Applicant: Bo Tao, Chino, CA (US)

(72) Inventor: Bo Tao, Chino, CA (US)

(73) Assignee: Bo Tao, Chino, CA (US)

(21) Appl. No.: 16/429,509

(22) Filed: Jun. 3, 2019

Related U.S. Application Data
(60) Provisional application No. 62/686,096, filed on Jun. 18, 2018.

(51) Int. Cl. A61K 8/06; A61C 19/06; A61J 1/06; A61J 1/20; A61M 3/02; A61K 8/22; A61K 8/25; A61K 8/73; A61K 8/81; A61K 8/55; A61K 8/9789; A61K 8/19; A61K 8/44; A61K 8/60; A61K 8/9794; A61Q 11/00; A61K 8/34; A61K 8/04

(52) U.S. Cl. CPC A61K 8/062; A61M 2210/0625; A61J 1/067; A61J 1/2093; A61M 3/0262; A61K 8/22; A61K 8/25; A61K 8/731; A61K 8/8176; A61K 8/55; A61K 8/9789; A61K 8/19; A61K 8/44; A61K 8/60; A61K 8/9794; A61Q 11/00; A61K 8/345; A61K 8/042; A61C 19/066

(57) ABSTRACT

A multi-component oral care formulation composed essentially of a first antiseptic composition and a second activating composition and multi-compartment delivery systems thereof, which are beneficial for stable storage and instant catalyzation and activation, are provided. The first composition comprises antiseptic compounds such as peroxygen compounds and colloidal silver as antiseptic agent and stabilizers and steric retarders that diminish the potential decomposition of the antiseptic compounds during storage. The second composition comprises peroxygen activation compounds that catalyze the breakdown of the antiseptic compounds in the first composition for more effective antiseptic action, at least one water soluble zinc compound that transforms the volatile sulfur compounds (VSC) into insoluble compounds, and chelating agents that effectively remove the tartar, and essential oils that help alleviate dry mouth through saliva stimulation, to promote overall oral health by providing multiple functions.

### BACKGROUND OF THE INVENTION

### 1. Field of the Invention

This invention relates generally to a pre-packaged, single-use, multi-component mouthwash in a multi-compartment delivery system for multi-function including antisepsis, anti-inflammation, anti-tartar, as well as the treatment and prevention of halitosis, gingivitis and periodontitis, which comprises a first antiseptic composition and a second activating composition which chemically interact and activate to rapidly accelerate and enhance the antisepsis, anti-tartar and halitosis treatment. More particularly, the present invention relates to a multi-compartment delivery system designed to stably store and conveniently deliver the multi-component oral care formulations. The first and the second compositions of the multi-component oral care formulations are maintained separately from one another until application. The first and the second compositions of the multi-component oral care formulations are dispensed simultaneously into the oral cavity by the multi-compartment delivery system, and when the user swishes the multi-component oral care formulations inside the oral cavity, the swishing action effectively mixes the two compositions so that and the peroxygen activation compounds in the second composition catalyzes and accelerates the breakdown of the antiseptic compounds in the first composition to achieve a more rapid and effective antiseptic action. The multi-compartment delivery systems are available in bulk or single-dose formats. This invention relates to methods of making and packaging such oral care mouthwash.

### 2. The Prior Art

Bad breath, halitosis and oral malodor are labels placed on an unpleasant smell or odor that results from the microbial degradation of oral organic substrates including volatile sulfur compounds (VSC) such as hydrogen sulfide, methyl mercaptan and dimethyl sulfide from the oral cavity. The most common cause of halitosis (bad breath) is related to deep carious lesions, periodontal disease, oral infections, peri-implant disease, pericoronitis, mucosal ulcerations, impacted food or debris and, mainly, tongue coating. In rare cases, bad breath can be caused by certain medical conditions. In dry mouth (xerostomia), the flow and composition of saliva may be affected. A lack of saliva can cause more bacteria than normal to build up in your mouth, as well as a change in the types of bacteria. A build-up of these in the mouth may lead to bad breath.

Several methods have been used to treat halitosis. These include mechanical removal of biofilm and microorganisms such as tongue cleansing in addition to brushing and flossing of teeth, chemotherapeutic reduction by using toothpastes and mouth rinses, and combining the mechanical and chemotherapeutic approaches.

The prior art discloses many methods of mouthrinse for the treatment of halitosis. U.S. Pat. No. 5,154,917 discloses a bottle having a removal cap and two separate compartments each having its own open spout, and the first compartment stores a red liquid at alkaline pH and comprising phenolphthalein and a yellow dye, and a blue liquid at acidic pH comprising a blue dye, where the two liquids combine when poured into a cup to form a liquid mixture of aqua color; a therapeutic oral hygiene medicament is included in one of said liquids in the bottle. U.S. Pat. No. 5,392,947 discloses a mouth rinse product comprising a first liquid containing hydrogen peroxide, a second liquid containing sodium bicarbonate filled in a dispensing container containing two separate compartments. U.S. Pat. No. 5,993,785 discloses a multi-component formulation suitable for use as a mouth rinse for the treatment of dental caries, which formulation comprises an aqueous calcium component, an aqueous phosphate component, whereby on mixing, the components form a solution supersaturated with respect to calcium phosphate(s). U.S. Pat. No. 64,099,992 discloses a two-component mouth rinse composition comprising a first component containing a zinc compound and a second component containing an oxidation compound, wherein the first and second components are stored separately and have to be pre-mixed prior to use these above-mentioned prior art patents concern the delivery of a liquid composition to the tooth, gums, or periodontal pockets. However, these compositions have the drawbacks of either not chemically activating, or using ineffective antisepsis activation compositions that do not kill or inhibit the growth of bacteria, thus not efficiently releasing all the oxygen radicals available in the antisepsis compounds quickly, making them ineffective when being used for 30 seconds or less in most cases. If the user chooses to swish the mouthwash for only a very short amount of time (i.e.: 10 to 15 seconds), the antiseptic compounds in the compositions will not have enough time to breakdown into oxygen radicals for the maximum antibacterial effect. However, long swishing times (60 seconds+) are often not possible, or at least very difficult for users to adhere to habitually, hence shorter mouthwash swishing time without jeopardizing the antiseptic effect is often desired.

In one aspect, the present invention is based upon the discovery that when a peroxide containing component and peroxide activating agents such as transitional metal salts or complexes, enzymes, electrolytes, alkaline, and mixtures thereof, which are normally incompatible with each other and combined for the first time, result in catalytic decomposition of hydrogen peroxide, and releases active radicals that rapidly kill off anaerobic bacteria colonization and prevent their multiplication by changing the microbial environment and breaking the protein chains. The catalytic decomposition depends upon the concentration of H₂O₂, the concentration of the catalyst, temperature, and pH of the reacting solutions.

The multi-component formulation and unit-dose delivery system deliver additional benefits to consumers, while also providing convenience. Such additional benefits preferably include improved shelf life, effective anti-inflammation, reduction of plaques, whitening of the teeth, no measuring, pre-mixing or wasting of compositions, and reduced required rinsing time and enhanced taste.

It is well known that compliance in a therapeutic regimen has been shown to be directly related to the length of the therapy and the frequency of dosage. Hence, one object of this invention is to provide a system which can more rapidly reduce oral malodor, gingivitis and periodontitis by preventing or reducing oral putrefaction without undesirable long mouth rinsing time (i.e.: 60+ seconds). Another object of this invention is to provide anti-halitosis and anti-gingivitis and anti-periodontitis mouth rinse systems and compositions which can be used both in the home or on-the-go by consumers. A further object of this invention is to provide desensitizing and anti-tartar effects during mouth rinsing, with no additional rinsing time.

### DETAILED DESCRIPTION OF THE INVENTION

### Definitions

The terms “components” and “compositions” are used interchangeably in this patent.

The terms “mouth rinse” and “mouthwash” are used interchangeably in this patent.

The present multi-component formulation can be presented in either liquid forms including solution, or suspension or emulsions, or semi-solid forms including gel, lotion or cream, or paste. Preferably, the present multi-component formulation is presented in gel forms that can be easily broken down into liquid upon being swished insider user's mouth.

An emulsion is a mixture of two or more liquids in which one is present as droplets, of microscopic or ultramicroscopic size, distributed throughout the other. Two liquids can form different types of emulsions. As an example, oil and water can form an oil-in-water emulsion, wherein the oil is the dispersed phase, and water is the dispersion medium; they can also form a water-in-oil emulsion, wherein water is the dispersed phase and oil is the external phase. Multiple emulsions are also possible, including a “water-in-oil-in-water” emulsion and an “oil-in-water-in-oil” emulsion.

The terms “gel” is defined as a transparent jelly-like substance which leaves a thin film of the active ingredient; while “lotion” or “cream” is defined as an emulsification of oil and water; and “paste” is non-transparent mixture. The term “swishable” means can be readily broken down or “breakable”.

All percentages, ratios and proportions herein are by weight, unless otherwise specified. All temperatures are in degrees Celsius (° C.) unless otherwise specified.

As used herein, “comprising” means that other steps and other ingredients which do not affect the end of result can be added. This term encompasses the terms “consisting of” and “consisting essentially of”.

Below is a brief description of the drawing:

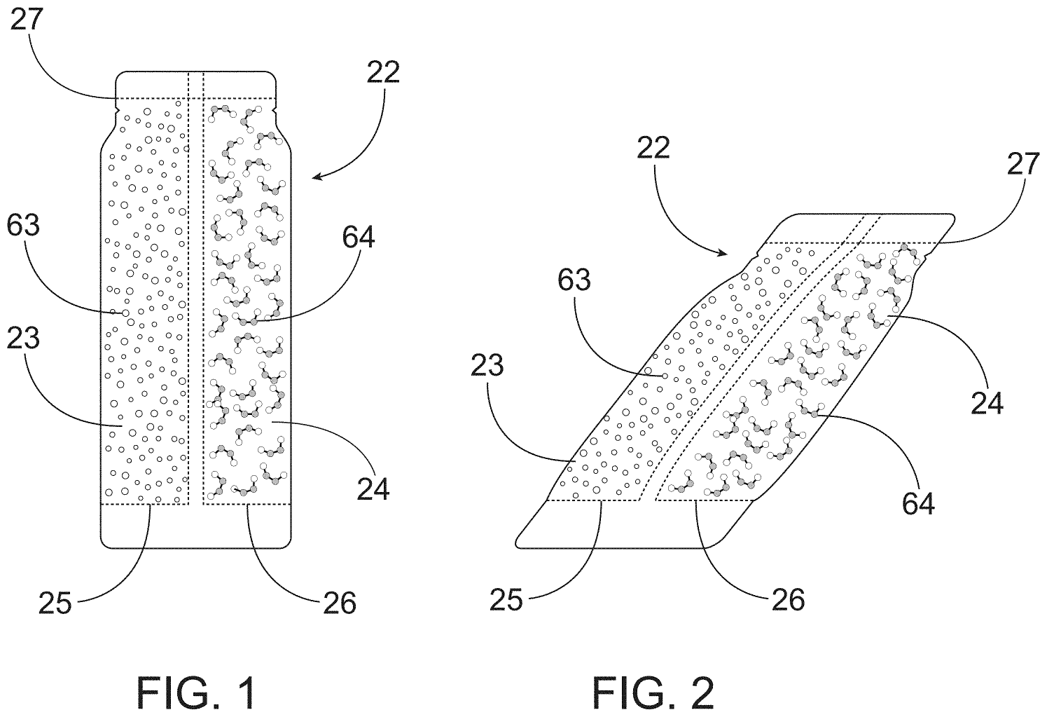

FIG. 1 and FIG. 2 show the top view and 45 degree angled view of a preferred embodiment of the present invention, which is a multi-compartment flexible and squeezable pouch comprising multi-layer laminate sheets heat-sealed to form separate compartments 25 and 26, whereby upon tearing open the perforation 27 and when said compartments are squeezed, the antiseptic component 24 and activating component 23 are urged out of said compartments directly into user's mouth, where the swishing action will effectively mix the antiseptic and activating components to catalyze the antiseptic/remineralization action.

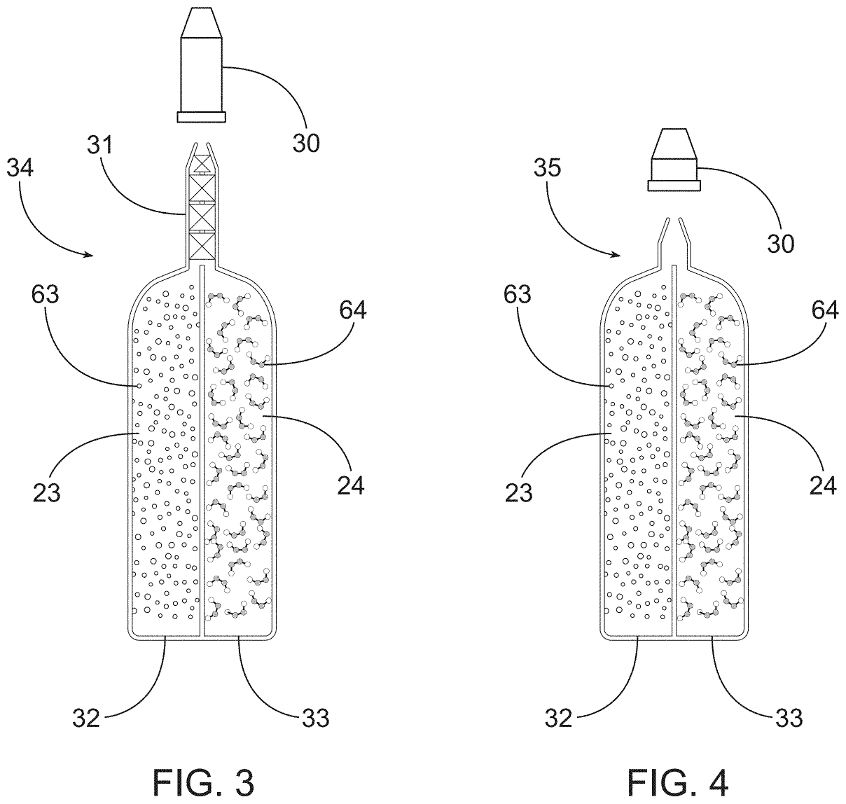

FIG. 3 and FIG. 4 show a flexible and squeezable plastic vial comprising a plurality of compartments 32 and 33 and an optional mixing application tip 31 with a cap 30 removably attached to said applicator tip 31, whereby when said cap 30 is removed from said applicator tip 31 and said compartments 32 and 33 are squeezed, the antiseptic component 24 and activating component 23 are urged out of said compartments 32 and 33 through said applicator tip 31's internal mixing elements to be mixed and dispensed into user's mouth.

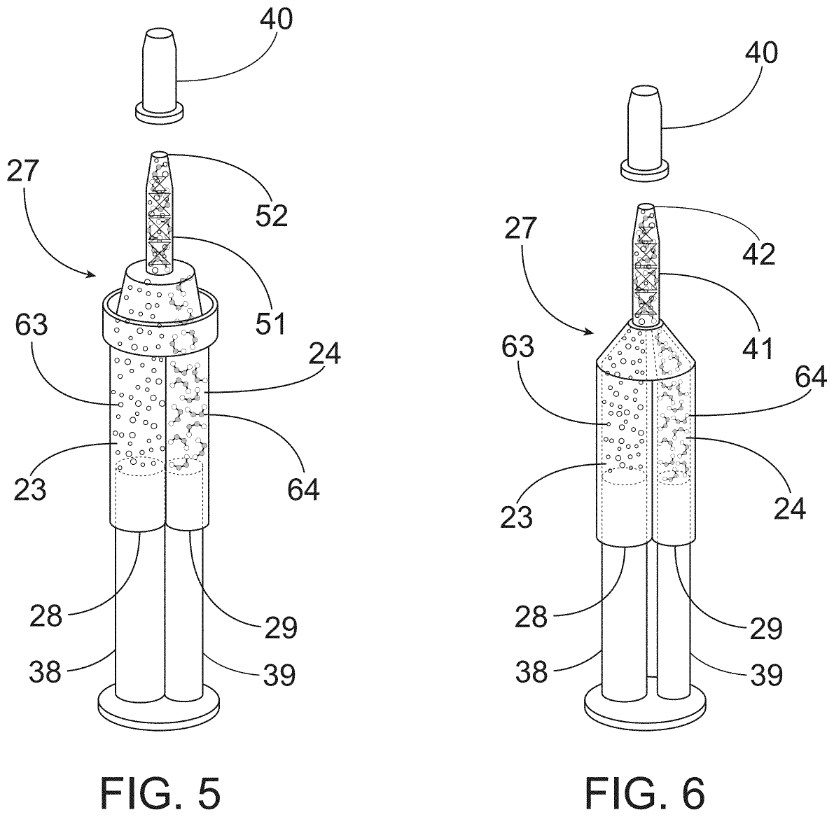

FIG. 5 and FIG. 6 show a rigid plastic multi-compartment container comprising a plurality of chambers 28 and 29 adjacent to each other; and a plurality of plungers 38 and 39 each received in a respective one of the plurality chambers 28 and 29 and operative to create a positive pressure sufficient to simultaneously actuate and drive said antiseptic component 24 and activating component 23 from the respective chambers into the downstream end of an integrated applicator tip 41 or removably attachable applicator tip 51, wherein the downstream end of said applicator tip 41 or 51 is provided with an outlet port 42 or 52; said applicator tip 41 or 51 includes internal mixing elements connected to the outlets of said separate chambers 28 and 29 for mixing and dispensing during use.

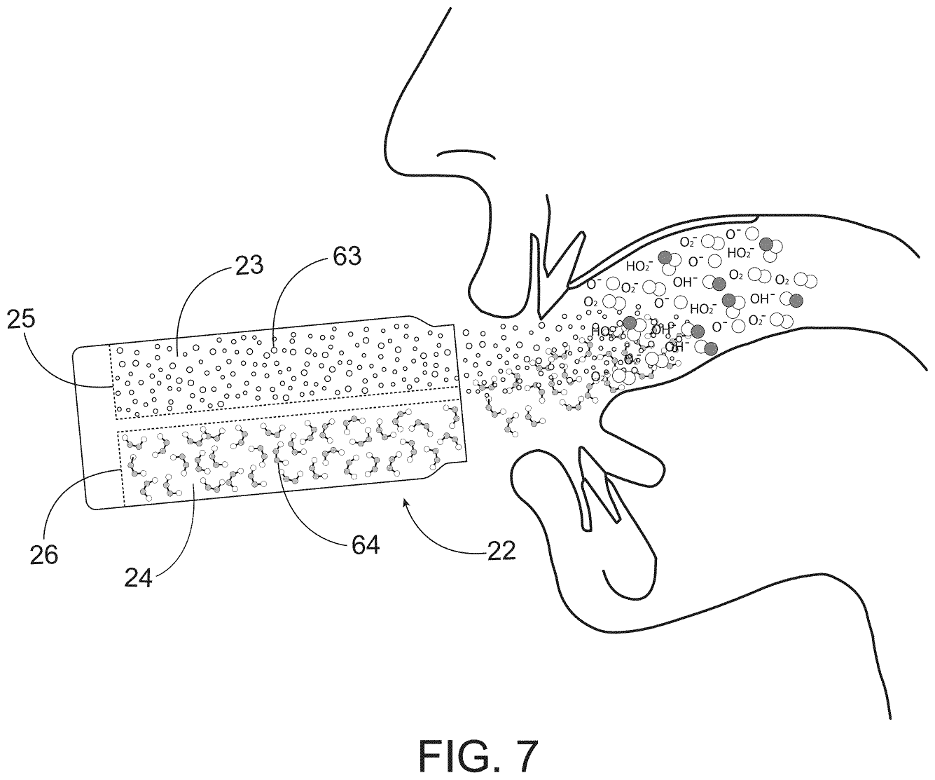

FIG. 7 shows a user tearing open a multi-compartment flexible and squeezable pouch comprising separate compartments 25 and 26, whereby when said compartments are squeezed, the antiseptic component 24 and activating component 23 are urged out of said compartments directly into the user's mouth, where the swishing action effectively mixes the antiseptic and activating components to catalyze the antiseptic/remineralization action.

Peroxygen compounds have been used for many years in mouth rinses because they are effective in curative and/or prophylactic treatments in control of various oral conditions. Peroxide-containing agents in the oral cavity exert a chemomechanical action generating thousands of tiny oxygen radicals produced by swishing and interaction with tissue and salivary enzymes. Peroxide mouth rinses and other oral preparations prevent colonization and multiplication of anaerobic bacteria known to be associated with periodontal disease as it can change the microbial environment and break the protein chains. In one aspect, Peroxygen compounds, for example, hydrogen peroxide, on their own, are mild topical anti-infective agents but aqueous solutions of high concentration of H₂O₂ would be detrimental if applied to the periodontium and to human tissue and teeth so that it is necessary to use a weak or mild solution of H₂O₂, preferably not exceeding 3% and/or to use the H₂O₂ in mouthrinse composition. Peroxygen compounds used in the compositions of the invention preferably include hydrogen peroxide, carbamide peroxide (urea peroxide), metal peroxides such as calcium peroxide, sodium peroxide, strontium peroxide, magnesium peroxide, and the salts of perbotate, persilicate perphosphate and percarbonate such as sodium perborate, sodium chlorite, potassium persilicate and sodium percarbonate. The most effective peroxygen compound for this invention is hydrogen peroxide. The useful range of hydrogen peroxide is between about 0.1% to about 3% by weight, which is equivalent to about 0.3% to 8.3% of carbamide peroxide. The preferred range for hydrogen peroxide is between about 1% to about 2% by weight (equivalent to 2.7% to 5.5% of carbamide peroxide).

Preferably, peroxygen compounds are stabilized by adding into the peroxygen compound stabilizers, selected from the group of EDTA and its salt, potassium stannate, sodium stannate, etidronic acid, phosphoric acid, sodium pyrophosphate. Optionally, peroxygen compounds are further stabilized by adding the steric retarder, essentially selected from the group of Polyvinylpyrrolidone (PVP), Poly(ethylene) Oxide; Poly(propylene) Oxide, Polyoxyethylene/polyoxypropylene block copolymer, and the mixtures thereof.

In one aspect, in the antiseptic component 24, there is a highly-effective peroxygen compound, such as hydrogen peroxide or carbamide peroxide, with additional antiseptic compounds such as nano-colloidal silver, stabilizers, thickening agents, etc.

The decomposition of hydrogen peroxide has been widely studied as a model reaction for its catalytic activity of various metal complexes and has also been studied as a catalase model. It has been known for about a century that the decomposition of H₂O₂ to H₂O and O₂ is drastically accelerated by a few different factors. In one aspect, the rate of hydrogen peroxide decomposition increases with rising temperature, concentration and pH. Particularly, the pH level of the peroxygen component is adjusted with a pH adjusting agent selected from sodium hydroxide, potassium hydroxide, ammonium hydroxide, triethylamine, and has a pH level ranging from 4.5 to 10.0.

In another aspect, decomposition is catalyzed by various compounds, including most transition metal compounds (e.g. manganese, Ferrous, Titanium, Cupric compounds and etc.). Certain metal ions, such as Fe²⁺ or Ti²⁺, can cause the decomposition to take a different path, with free radicals such as (HO.) and (HOO.) being formed. Non-metallic catalysts include potassium iodide, which reacts particularly rapidly. Hydrogen peroxide can also be decomposed biologically by enzyme catalase. In a further aspect, peroxygen compounds can achieve some antisepsis unaided if used for long periods of time, but for truly effective applications, it requires chemical activation in one way or another.

Therefore, the present invention provides a multi-component mouthwash gel in a multi-compartment delivery system for antisepsis, anti-inflammation, as well as the treatment and prevention of halitosis, gingivitis and periodontitis, wherein a first antisepsis component 24 comprises a peroxygen compound 64, such as hydrogen peroxide, sodium chlorite or sodium perborate, stabilizers, and thickening agents, and separate second activating component 23 comprises at least one activating compound or composition 63 and at least one soluble metal salt essentially selected from zinc acetate, zinc chloride, zinc citrate, zinc gluconate, zin lactate, zinc sulfate, and the mixture thereof that can transform VSC into insoluble compounds and thus reduce malodor emanated from the VSC in oral cavity and/or desensitizing agents and/or anti-inflammatory agents. The activation compound/complex 63 is defined here as a transition metal salt or a transition metal complex consisting of a transition metal compound consisting of Manganese, Ferrous, Cupric, Titanium compounds and an chelating agent selected from disodium ethylenediaminetetraacetic acid (EDTA), trisodium EDTA, tetrasodium EDTA, and etc., alone or mixtures thereof, and/or pH-enhancing agents, and/or electrolytes, and/or bioactive enzymes and etc., alone or mixtures thereof, which is capable of activating the peroxygen compound in a manner that produces active oxygen radicals for efficacious antiseptic actions. When the two components are mixed together, the activating compound or composition 63 included in the activating component 23 activates the peroxygen compound 64 in the antisepsis component 24, and accelerates the release of active oxygen radicals for accelerated antisepsis action, and meanwhile, the compositions also desensitize the teeth and/or reduce tooth plaque or calculus and/or anti-inflammation.

In one aspect, the amount of peroxygen activation compound/complex 63 (such as transition metal compound/complex, and/or pH-enhancing agents, and/or electrolytes, and/or bioactive enzymes and etc., alone or mixtures thereof) present in the activating component 23 of the present invention will vary depending upon the amount of peroxygen compound 64 incorporated in the antiseptic component 24. To achieve an effective activation, the amount of transition metal ions is ranging from about 0.05% to 5% and preferably, from about 0.2% to 2%.

Preferably, the said second activating component 23 and said first antisepsis component 24 are both gels based on water soluble polymers, such as natural, semi-synthetic and synthetic polymers, including but not limited to guar gum, karya gum, xanthan gum, pectin, starch, locust bean gum, tara gum, gelatin, pullulan, maltodextrins, chitosan derivatives, sodium alginate, polyethylene glycol (PEG), polyvinylpyrrolidone (PVP), polyvinyl alcohol, polyacrylic acid, salts of polyacrylic acid including sodium salt, potassium salt and ammonium salt, poly(meth)acrylic acid and its derivatives, hydroxypropylmethyl cellulose (HPMC), hydroxyethyl cellulose (HEC), hydroxypropyl cellulose (HPC), carboxymethyl cellulose (CMC), sodium carboxymethyl cellulose (Na-CMC), alone or mixtures thereof. The said activating and antisepsis components comprise mainly about 0.01% to 10% of the said water-soluble polymers by weight of the total gel weight and preferably about 0.1% to 5% by weight. These polymers are also called gel-forming polymers. It has been demonstrated that the properties of the gel-forming polymers play a critical role in the formulations' final performance. The physicochemical properties of the gel-forming polymers should be considered when formulating. These properties include compatibility of the gel-forming polymers with the active ingredients (both peroxygen compounds and activating compounds), compatibility with solvents, the polymorphic nature and synergistic properties of the gelatin-forming polymers selected and temperature sensitivity.

It has been found there is a synergistic effect with certain combinations of polysaccharide gums and thus gels can be formed even though one or more of the gums does not form a gel on its own. In the present invention, the said activating and antisepsis components comprise blends of the above-mentioned polysaccharide gums, especially with natural vegan gums such as xanthan gum, pectin, locust bean gum, tara gum, agar, carrageenan gum, cassia gums, and etc. The blends or combinations of the gums offer additional advantages in said components such as good mechanical properties to prevent spilling during dispensing and for swishing inside mouth during use. Preferably, the ratios of xanthan gum, galactomannan or glucomannan gum each lie within the range of from 1:5 to 5:1. Particularly, soft gels with desirable textures and/or strength have been found to be formed at a ratio of xanthan gum:carob locust bean gum of within the range of 1:2 to 2:1. Optionally, the blends or combinations may comprise three gel-forming polymers and the rations of xanthan gum, carob gum or cassia or konjac gum of within the range of 1:2 to 2:1 to 2 and especially 1:1:1. When tara gum is used a higher proportion is desirable and the preferable ration of gum is 3:2:2.

In another aspect, the said activating component 23 comprises at least one chelating agent as anti-tartar agents. The said chelating agents are selected from disodium ethylenediaminetetraacetic acid (EDTA), trisodium EDTA, tetrasodium EDTA, and etc., alone or mixtures thereof. The chelating agents exert as chelators to not only complex the transition metal ions but also complex the calcium ions to prevent it from forming calculus thus providing anti-tartar effect. To ensure both effective activation and anti-tartar activities, the amount of the chelating agents is ranging from 0.1% to 10% and preferably from 0.5% to 5% by weight of the total composition weight.

Optionally, the said activating component 23 comprises desensitizing agents selected from the group of sodium citrate, potassium citrate, potassium nitrate, and etc. The amount of the said desensitizing agent is ranging from 0.1% to 10% and preferably from 0.5% to 5% by weight of the total composition weight.

In a further aspect, the said activating component 23 may comprise anti-inflammatory agents. The said inflammatory agents are essentially selected from but not limited to the group of herbal and aromatic oils, more preferably from aniseed oil, basil oil, camphor oil, cannabidiol oil, cedarwood oil, cinnamon oil, clove oil, coconut oil, eucalyptus oil, frankincense oil, lemon oil, lime oil, lemongrass oil, orange oil, peppermint oil, rosemary oil, tea tree oil, thyme oil, and the mixture thereof, and the group of herbal extracts, essentially selected from arnica flower extract, aloe vera extract, clove bud extract, cinnamon bark extract, ginger extract, peppermint leaf extract, and the mixture thereof. Additional ingredients that are incorporated include sweetening and flavoring agents, coloring agents, saliva-stimulating agents, surfactants and thickening agents.

Additionally, surfactants can be used to disperse the peroxygen activation compound/complex and improve the diffusion of the free oxygen radicals and thus achieve effective antiseptic results during the short rinsing time of the multi-component mouthrinse.

Preferably, at least one humectant such as glycerin, propylene glycol, polyethylene glycol (PEG) 200, 400, 600, polymer PEGs, copolymer of polyethylene oxide and polypropylene oxide, and mixtures thereof, is used when making the said vegan gelatin. The amount of the humectant can vary from 0.1 to 40%, preferably, from 0.5% to 25% by weight.

The vehicles used to prepare the individual components of the multi-component oral care formulations of the present invention are similar. Both the first activating component 23 and the antiseptic component 24 are made of vegan gelatin-forming polymers such as natural, semi-synthetic and synthetic polymers, including but not limited to guar gum, karya gum, xanthan gum, tara gum, locust bean gum, pectin, starch, gelatin, pullulan, maltodextrins, chitosan derivatives, sodium alginate, polyethylene glycol (PEG), polyvinylpyrrolidone (PVP), polyvinyl alcohol, polyacrylic acid, salts of polyacrylic acid including sodium salt, potassium salt and ammonium salt, poly(meth)acrylic acid and its derivatives, hydroxypropylmethyl cellulose (HPMC), hydroxyethyl cellulose (HEC), hydroxypropyl cellulose (HPC), carboxymethyl cellulose (CMC), sodium carboxymethyl cellulose (Na-CMC), alone or mixtures thereof; with additional incorporated ingredients including sweetening and flavoring agents, coloring agents, saliva-stimulating agents, and thickening agents. The vehicle used to prepare the compositions of the two components includes water and/or a suitable humectant such as glycerin, propylene glycol, polyethylene glycol, or any suitable mixture thereof. Water is preferred as a humectant in the practice of the present invention.

In a further aspect, the two components are maintained separately until application to the mouth wherein the peroxygen activation compound/complex 63 in the activating component 23 interacts with the peroxygen compound 64 in the antiseptic component 24 to accelerate the rapid release of active oxygen radicals from the peroxygen compound 64, such rapid release being highly effective for antisepsis as illustrated in FIG. 7. FIG. 7 can also illustrate the use of this multi-component oral care system for the purpose of rem ineralization by generating the precursors for in-situ hydroxyapatite formation on teeth through the inclusion of calcium complex and phosphate complex in component 23 and 24. The present invention offers the advantages that the premature breakdown of the peroxygen compound 64 is avoided, and the active oxygen radicals are generated quickly and in large quantities during use thereby facilitating shorter rinsing time, better efficacy and improved user experience.

In a further aspect, the present invention offers a method of applying the multi-component oral care system for a multi-component desensitization or in-situ remineralization formulation. The method of application consists of:

a) dispense the activating component 23 into an oral cavity, then immediately dispense the antiseptic component 24 into the oral cavity; or,

b) dispense the antiseptic component 24 into an oral cavity, then immediately dispense the activating component 23 into the oral cavity.

Benefits of the multi-component oral care formulations and multi-compartment delivery system when compared to existing mouthrinse formulations include:
The contact of the activating component 23 and the antiseptic component 24 instantly activates and catalyzes the decomposition of peroxygen compound 64 antiseptic component 24;
Greatly reduced rinsing time to achieve the same or better antiseptic, anti-halitosis, desensitization and remineralization result;
Separation of the peroxygen compound (active antiseptic ingredient) 64 and the peroxygen activation compound 63 improves the stability of the compositions and shelf-life;

The following examples are further illustrative of the present invention, but it is understood that the invention is not limited thereto. All amounts and proportions referred to herein and the appended claims are by weight.

### EXAMPLE 1

One set of example formulations of the first antiseptic component and the second activating component are made according to the present invention, containing mixtures of, HPMC and Xanthan Gum, Carob Gum as gel-forming polymers, Hydrogen Peroxide as antiseptic agent; transition metal salt, manganese gluconate as peroxide activating agent, PVP as surfactant, etc.

Composition ranges of various ingredients used in the first antiseptic composition

| Ingredients | % by weight |
|---|---|
| DI water | 54.7 |
| Glycerin | 20 |
| Hydrogen Peroxide 50% Solution | 6 |
| Fumed Silica | 6 |
| Hydroxypropyl Methylcellulose | 5 |
| Potassium Citrate | 2 |
| PVP K-30 | 2 |
| D,L-Menthol | 1 |
| Xanthan Gum | 1 |
| Sodium Polyphosphate | 0.8 |
| Peppermint oil | 0.8 |
| Sodium Saccharin | 0.4 |
| Potassium Stannate | 0.3 |
| Nano-colloidal Silver | 20 ppm |

Composition ranges of various ingredients used in the second activation composition

| Ingredients | % by weight |
|---|---|
| DI water | 66.9 |
| Glycerin | 20 |
| Potassium Citrate | 2 |
| Disodium EDTA | 2 |
| Zinc Lactate Gluconate | 1.8 |
| Tetrasodium EDTA | 1.5 |
| D,L-Menthol | 1 |
| Coconut Oil | 1 |
| Manganese Gluconate | 0.8 |
| Flavorant | 0.8 |
| Xanthan Gum | 0.7 |
| Carob Gum | 0.7 |
| Potassium Sorbate | 0.3 |
| Sodium Saccharin | 0.3 |
| Methyl Salicylate | 0.2 |
| Colorant | Trace |

Viscosity of the formulation was measured by Brookfield rheometer with spindle 51 at 200 rpm. The viscosity of the antiseptic component was 90 Centipoises (cps), while the viscosity of the activating component was 85 cps.

### EXAMPLE 2

Another set of example formulation of the first antiseptic component and the second activating component is made according to the present invention, containing mixtures of Xanthan Gum, Carob Gum as gel-forming polymers, Hydrogen Peroxide as antiseptic agent; transition metal salt, manganese gluconate as peroxide activating agent, PVP as surfactant, and etc.

Composition ranges of various ingredients used in the first antiseptic composition

| Ingredients | % by weight |
|---|---|
| DI water | 68.3 |
| Glycerin | 20 |
| Hydrogen Peroxide 50% Solution | 3 |
| Potassium Citrate | 2 |
| PVP K-30 | 2 |
| D,L-Menthol | 1 |
| Sodium Polyphosphate | 0.8 |
| Peppermint oil | 0.8 |
| Xanthan Gum | 0.7 |
| Carob Gum | 0.7 |
| Sodium Saccharin | 0.4 |
| Potassium Stannate | 0.3 |
| Nano-colloidal Silver | 20 ppm |

Composition ranges of various ingredients used in the second activation composition

| Ingredients | % by weight |
|---|---|
| DI water | 66.7 |
| Glycerin | 20 |
| Potassium Citrate | 2 |
| Disodium EDTA | 2 |
| Zinc Lactate Gluconate | 1.8 |
| Tetrasodium EDTA | 1.5 |
| D,L-Menthol | 1 |
| Coconut Oil | 1 |
| Manganese Gluconate | 1 |
| Flavorant | 0.8 |
| Xanthan Gum | 0.6 |
| Carob Gum | 0.6 |
| Fruit Pectin | 0.2 |
| Potassium Sorbate | 0.3 |
| Sodium Saccharin | 0.3 |
| Methyl Salicylate | 0.2 |
| Colorant | Trace |

Viscosity of the formulation was measured by Brookfield rheometer with spindle 51 at 200 rpm. The viscosity of the antiseptic component was 80 Centipoises (cps), while the viscosity of the activating component was 90 cps.

### EXAMPLE 3

Another set of example formulation of the first antiseptic component and the second activating component is made according to the present invention, containing mixtures of Xanthan Gum, Tara Gum and Fruit Pectin as gel-forming polymers, Hydrogen Peroxide as antiseptic agent; transition metal salt, manganese gluconate as peroxide activating agent, PVP as surfactant, and etc.

Composition ranges of various ingredients used in the first antiseptic composition

| Ingredients | % by weight |
|---|---|
| DI water | 67.5 |
| Glycerin | 20 |
| Hydrogen Peroxide 50% Solution | 4 |
| Potassium Citrate | 2 |
| PVP K-30 | 2 |
| D,L-Menthol | 1 |
| Sodium Polyphosphate | 0.8 |
| Peppermint oil | 0.8 |
| Xanthan Gum | 0.72 |
| Tara Gum | 0.48 |
| Sodium Saccharin | 0.4 |
| Potassium Stannate | 0.3 |
| Nano-colloidal Silver | 20 ppm |

Composition ranges of various ingredients used in the second activation composition

| Ingredients | % by weight |
|---|---|
| DI water | 66.7 |
| Glycerin | 20 |
| Potassium Citrate | 2 |
| Disodium EDTA | 2 |
| Zinc Lactate Gluconate | 1.8 |
| Tetrasodium EDTA | 1.5 |
| D,L-Menthol | 1 |
| Coconut Oil | 1 |
| Manganese Gluconate | 1 |
| Flavorant | 0.8 |
| Xanthun Gum | 0.6 |
| Tara Gum | 0.4 |
| Fruit Pectin | 0.2 |
| Potassium Sorbate | 0.3 |
| Sodium Saccharin | 0.3 |
| Methyl Salicylate | 0.2 |
| Colorant | Trace |

Viscosity of the formulation was measured by Brookfield rheometer with spindle 51 at 200 rpm. The viscosity of the antiseptic component was 75 Centipoises (cps), while the viscosity of the activating component was 85 cps.

A method of using the multi-compartment delivery system for a multi-component mouth rinse composition comprises: tear along perforation of a single-use multi-compartment pouch, or alternatively, open the cap of a single-use squeezable multi-compartment vial, then dispense both compositions inside the two compartments of said pouch directly into mouth, swish the contents around thoroughly for 30 seconds to mix and catalyze the antiseptic and/or other functional actions (such as rem ineralization and anti-sensitivity), then spit out the mixture and leave a small amount of residues inside mouth without rinsing afterward.

What is claimed is:

1. A multi-component oral care formulation composed essentially of at least two components, wherein said first antiseptic component consisting of antiseptic agents essentially selected from peroxygen compounds such as hydrogen peroxide, carbamide peroxide, sodium peroxide, calcium peroxide, calcium peroxide, sodium perborate, chlorine dioxide, sodium percarbonate, and sodium chlorite, stabilized with at least one stabilizer, and more particularly, at least one radical scavenger for minimizing peroxygen compound decomposition during storage; wherein said second activating component comprising water-soluble transition metal compounds or enzymes or electrolytes to catalyze the breakdown of the antiseptic agents in said first component to promote the antiseptic effect, water soluble zinc compounds to further diminish bad tastes and reduces tartar buildup, and essential oils as additional antibacterial agents and anti-inflammatory agents presented in an oil-in-water emulsion preparation. The antiseptic component and the activating component are stored separately in a multi-compartment container for maximum stability, and when the multi-components are dispensed and mixed inside user's oral cavity during use, free radicals are released rapidly from the antiseptic compounds for greatly enhanced antiseptic and oral care functions.

2. The multi-component oral care formulations as claimed in claim 1 are presented in flowable liquid, or emulsion, or gel form, or lotion, or cream, or paste, preferably in gel or paste forms that are “swishable” in mouth, with both components having the same or similar viscosity ranging from 10 to 10,000 Centipoises (cps).

3. The antiseptic component as claimed in claim 1 wherein said antiseptic agent stabilizer is essentially selected from the group of Edetic acid and disodium EDTA, potassium stannate, sodium stannate, etidronic acid, sodium pyrophosphate, and the mixtures thereof.

4. The antiseptic component as claimed in claim 1 wherein said the steric retarder is essentially selected from the group of Polyvinylpyrrolidone (PVP), Poly(ethylene) Oxide; Poly(propylene) Oxide, Polyoxyethylene/polyoxypropylene block copolymer, and the mixtures thereof.

5. The antiseptic component as claimed in claim 1 when presented in non-flowable gel or paste, wherein said component contains at least one stable thickener present in amount from about 0.5% to about 40.0% by weight of the total component weight, which is essentially selected from the group of Polyvinylpyrrolidone (PVP), Poly(ethylene) Oxide, Poly(propylene) Oxide, Polyoxyethylene/polyoxypropylene block copolymer, Hydroxymethyl Cellulose, Hydroxyethyl Cellulose, Hydroxypropyl Cellulose, fumed silica, and the mixture thereof.

6. The antiseptic component as claimed in claim 1 further comprises of a mixture of propylene glycol/glycerin as humectant from about 5% to about 40%, to retain the moisture of the formulation. The antiseptic component as claimed in claim 1 wherein said component's pH level is adjusted with a pH adjusting agent selected from sodium hydroxide, potassium hydroxide, ammonium hydroxide, triethylamine, and has a pH level ranging from 4.5 to 10.0.

8. The second activating component as claimed in claim 1, wherein said activating agents are essentially selected (but not limited to) from the group of transition metal compounds, enzymes, electrolytes, or the mixture thereof, to effectively activate peroxygen compounds in the antiseptic component.

9. The second activating component as claimed in claim 1, wherein water-soluble zinc compounds essentially selected from zinc acetate, zinc chloride, zinc citrate, zinc gluconate, zin lactate, zinc sulfate, and the mixture thereof are further present.

10. The second activating component as claimed in claim 1 wherein said component is further comprises essential oils that have antibacterial and/or anti-inflammatory and/or anti-irritant effects, essentially selected from but not limited to the group of herbal and aromatic oils, more preferably from aniseed oil, basil oil, camphor oil, cannabidiol oil, cedarwood oil, cinnamon oil, clove oil, coconut oil, eucalyptus oil, frankincense oil, lemon oil, lime oil, lemongrass oil, orange oil, peppermint oil, rosemary oil, tea tree oil, thyme oil, and the mixture thereof.

11. The second activating component as claimed in claim 1, wherein said component comprises herbal extracts, essentially selected from arnica flower extract, aloe vera extract, clove bud extract, cinnamon bark extract, ginger extract, peppermint leaf extract, and the mixture thereof.

12. The second activating component as claimed in claim 1, wherein said component is comprises nano-colloidal silver present in amount of 0.5 to 60 ppm.

13. The second activating component as claimed in claim 1, if presented in cream containing oil-in-water, wherein said component is made by mixing a water phase and oil phase under high speed stirring or homogenizing to form an oil-in-water emulsion to improve the solubility and absorption of the essential oils.

14. The activating component as claimed in claim 1, when presented in gel or paste form, wherein the said component further comprises a thickening agent, essentially selected from the group of hydrophilic adhesive polymers, including but not limited to ammonium acryloyl dimethyltaurate/vinylpyrrolidone (VP) compolymer, polyacrylic acid, starch, hydroxypropyl methyl cellulose, hydroxyethyl cellulose, gelatin, xanthan gum, carob locust bean gum, tara gum, fruit pectins and the mixture thereof, in amount of from about 0.05% to about 10%.

15. The second activating component as claimed in claim 1, wherein the pH is adjusted to a neutral to slightly alkaline values ranging from 5.0 to 8.5, with a pH adjusting agent essentially selected from sodium hydroxide, potassium hydroxide, ammonium hydroxide, triethylamine.

16. A multi-compartment delivery system with any ratio from 1:10 to 10:1, wherein the first antiseptic component and the second activating component are separate during storage.

17. The multi-compartment delivery system as defined in claim 16, wherein the delivery container can be in the form of:
a flexible and squeezable pouch comprising multi-layer laminate sheets heat-sealed to form separate compartments; or
a flexible and squeezable plastic vial comprising a plurality of compartments; or
a rigid plastic container comprising a plurality of chambers adjacent to each other; and a plurality of plungers each received in a respective one of the plurality chambers and operative to create a positive pressure sufficient to simultaneously actuate and drive said antiseptic and activating components from the respective chambers into the downstream end of an applicator tip, wherein the downstream end of said applicator tip is provided with an outlet port; and
an integrated or removably attachable applicator tip including internal mixing elements connected to the outlets of said separate compartments for mixing and dispensing during use; and
a cap removably attached to said multi-compartment delivery system and sealing said applicator tip, whereby when said cap is removed from said pouch and said compartments are squeezed, the antiseptic and activating components are urged out of said compartments through said applicator tip's internal mixing elements to be mixed and dispensed.
